(19) United States
(12) Patent Application Publication
(10) Pub. No.: US 2026/0157645 A1
(43) Pub. Date: Jun. 11, 2026

(54) WEARABLE DEVICE FOR MEASURING BLOOD PRESSURE

(71) Applicant: HINLAB, NEUILLY-SUR-SEINE (FR)

(72) Inventor: Denys-Michel DE BEGON DE LAROUZIERE DE MONTLOSIER, NEUILLY-SUR-SEINE (FR)

(73) Assignee: HINLAB, NEUILLY-SUR-SEINE (FR)

(21) Appl. No.: 19/128,758

(22) PCT Filed: Nov. 10, 2023
(86) PCT No.: PCT/EP2023/081419

§ 371 Date: May 9, 2025

(30) Foreign Application Priority Data
Nov. 10, 2022 (EP) 22206754.8

(51) Int. Cl. A61B 5/022; A61B 5/00

(52) U.S. Cl. CPC A61B 5/02233; A61B 5/681; A61B 2560/0223; A61B 2560/0456; A61B 2562/0247

(57) ABSTRACT

A wearable device for measuring blood pressure of a subject includes a control unit including a pump and first connection configured to allow a fluid to flow between the pump and a bladder; a band device configured to circle a body portion of the subject, the band device including a bracelet including the bladder; and a docking station configured to removably secure the control unit. A first extremity of the bracelet is connected to a first lateral portion of the docking station and a second extremity of the bracelet is connected to a second lateral portion of the docking station, the docking station being interposed between the first extremity and the second extremity to close the band device.

### FIELD

The present invention pertains to the field of devices and methods for physiological parameter estimation. In particular, the invention relates to a wearable device for measuring blood pressure and to the band device and control unit that are included in it.

### BACKGROUND

Traditional sphygmomanometers comprise an inflatable cuff configured to be wrapped around an arm of a subject. The cuff may be inflated and deflated using either a manual pump or an automatized pump.

The blood pressure may be obtained by determining the systolic and diastolic pressures. To estimate the systolic pressure, the cuff has to be inflated, so as to occlude the brachial artery, while to determine the diastolic pressure, the cuff has to be deflated until blood flow in the brachial artery is restored.

Both systolic and diastolic pressures of the subject may be estimated either with an auscultatory technique or an oscillometric technique. The first technique is based on the Korotkoff sounds recorded with a stethoscope during the cuff inflations and deflations. The second technique estimates the systolic and diastolic pressures based on the amplitude of the pressure oscillations recorded within the cuff during deflation or inflation.

Several issues with traditional sphygmomanometers have arisen. Indeed, sphygmomanometers are very frequently used during auscultation at a doctor's office or in a hospital. Due to frequent manipulations, the cuff fabric tends to wear out quickly and it is necessary to change the whole device, even though the device electronics and/or pump is still functioning. This tends to be quite expensive and to create a lot of waste. Moreover, the medical staff is required to disinfect the device between to patients'auscultation. Disinfection of sphygmomanometers can be very complex because of the embedded electronics and pump that can be damaged by cleaning products or water.

A lack of adaptability to patients'different needs and morphologies has also been identified in traditional sphygmomanometers. For instance, a child has smaller limbs and a typical cuff is not adapted to the size of a child's limb. Therefore, the blood pressure measurements may be of lesser quality because the cuff is badly positioned.

The problem which the invention seeks to solve is to develop a sphygmomanometer that is more adaptable to different morphologies of patients, easier to clean and more environmentally friendly.

### SUMMARY

To solve this problem, the Applicant proposes a wearable device for measuring blood pressure of a subject comprising:
a control unit comprising:
   a housing;
   pumping means within said housing; and
   first connection means in fluidic connection with the pumping means, said first connection means being configured to allow a fluid to flow between the pumping means and an external environment of the housing; and
a band device configured to circle a body portion of the subject when in a closed state, said band device comprising:
   a bracelet comprising a bladder configured to be inflated and deflated to exert a pressure on the body portion; and
   a docking station configured to removably secure the control unit, said docking station including second connection means configured to co-operate with the first connection means to allow said pumping means to inflate or deflate the bladder.

The control unit is configured to determine blood pressure using the pressure exerted by the bladder on the body portion.

The invention is characterized in that a first extremity of the bracelet is connected to a first lateral portion of either one of the docking station and the control unit, and a second extremity of the bracelet is connected to a second lateral portion of the docking station, said first lateral portion and second lateral portion facing each other. At least one of the first lateral portion and second lateral portion forms a connecting portion, said connecting portion including the second connection. As a result, the docking station is interposed between the first extremity and the second extremity in the closed state.

In other words, the invention proposes a wearable device where all the electronics and pumping means are gathered and protected inside the control unit housing, while the band device only comprises the bladder. Advantageously, the control unit is removable from the band device and can be attached or detached from the band device at will.

By removing the control unit, the band device can therefore be cleaned without any risk of damaging the electronics. Moreover, when the cuff fabric is worn out, it is possible to only change the band device while keeping the control unit, which represents a real economy of resources. Therefore, the wearable device lasts longer and is more environmentally friendly.

Advantageously, it is also possible to adapt band devices of different sizes to the control unit to better fit the patient's morphology. For instance, it is possible to use a smaller cuff for children so that the wearable device better fits around the child's limb or bigger cuffs for overweight people. The resulting blood pressure measurement is more reliable because it circles the whole limb and because it is better positioned on said limb.

According to another embodiment, it is also possible to adapt different control units to the band device. The different control units may comprise different features such as additional sensors to measure other physiological parameters.

Therefore, the invention also relates to a kit comprising:
a control unit, and
a range of band devices with bracelets of different sizes to adapt to different types of body parts.

The invention also relates to a kit comprising:
a range of control units, said control unit further comprising at least one sensor configured to measure at least one physiological parameter, and
a band device.

The additional sensors may be an optical sensor, a temperature sensor, a heat flux sensor, a position sensor, an accelerometer, a pressure transducer, an ultrasound sensor, a gyroscopic sensor, an electrical sensor, such as for instance an electrical sensor comprising electrodes, a tomographic sensor, an acoustic sensor, a magnetometer, a humidity sensor, or even an impedance sensor. They may be used to calculate parameters such as the oxygen saturation, the heart rate, the respiratory rate, the blood pressure trend, the heart rate variability, the cardiac output, the skin temperature, the body temperature, the body composition . . . .

In practice, the control unit housing comprises a bottom side. Said bottom side includes in particular a sensor configured to come in contact with the body portion. To better accommodate the control unit, the docking station comprises a bottom wall configured to come in contact with the skin, framed by two lateral walls comprising the second connection means. The bottom wall and lateral walls form a reception site configured to receive the control unit. The sensor can be put in contact with the skin thanks to a through hole in the bottom wall of the docking station. More specifically, the control unit housing includes a protrusion to encapsulate at least sensor, and make sure it can be applied to the skin of the patient. The protrusion containing the sensor can therefore be inserted in the through hole.

As previously explained, the cooperation between the control unit and the band device is ensured by the cooperation between the first connection means present on the control unit and the second connection means present on the docking station. On the other hand, the docking station also cooperates with the extremities of the bracelet to form the band device and allow to circle a subject's body portion. Advantageously, the second connection means may be configured to also attach at least one of the first extremity and second extremity of the bracelet to the docking station. Therefore, the second connection means have both the function of connecting the docking station with to the control unit and connecting the docking station to the bracelet, which improves the compactness of the wearable device.

Advantageously, the second connection means include at least one sealing element comprising:
a tube-shaped body configured to cross through the connecting portion,
a first end configured to co-operate with the first connection means to establish a fluid-proof connection, and
a second end configured to co-operate with the bladder to establish a fluid-proof connection.

In other words, the sealing element puts in fluidic connection the control unit with the band device and more particularly the pump with the bladder. Advantageously, the sealing element absorbs a large majority of the efforts at the connection and protects the elements included in the housing such as the pump or a pressure sensor.

In another embodiment, the connecting portion further includes at least one securing element. The securing element and the sealing element are configured to at least partially be inserted in a recess arranged in the connecting portion. This improves the compactness of the wearable device. The securing element is configured to keep the sealing element in place and to strengthen the fluid-proof connections against impacts or wear. More precisely, the securing element may comprise an upper part and a lower part. The upper part and lower part are configured to co-operate to accommodate the tube-shaped body of the sealing element and to maintain one of the first extremity and second extremity of the bracelet at the same time. The upper part and lower part may be maintained together using fixing elements.

In practice, according to an embodiment, the bladder comprises an opening configured for the insertion of the sealing element. The second end is inserted inside this opening by deforming the flaring shape. Once inserted, the deformed second end reverts to its original flaring shape due to its high elasticity. Because the opening dimensions is smaller than the flaring shape dimensions, the latter cannot be easily removed from inside the bladder. Moreover, in the inserted position, the flaring shape is pressed against said opening to ensure fluid-proofness of the connection.

The first end of the sealing element is configured to be connected to the control unit. To that end, the first end may co-operate with several types of first connection means such as male-type first connection means or female-type first connection means.

In the first case, the first end forms inner and outer contact surfaces. The first end is arranged between the first connection means and the securing element. The first connection means come in contact with the inner contact surface of the first end, while the securing element comes in contact with the outer contact surface. The first end is therefore sandwiched and maintained into place between the securing element and the control unit, thus ensuring fluid-proofness of the connection.

In the second case, the first end forms a contact surface configured to be maintained inside said first connection means. It may be inserted in force in the connection means of the control unit. Preferably the first end is deformable to be inserted in the first connection means. Once inserted, the deformed first end reverts to its original shape due to its high elasticity. Because the first connection means dimensions are smaller than the first end dimensions, the first end cannot be easily dislodged. More precisely, the flaring shape is pressed against the first connection means to ensure fluid-proofness of the connection.

According to another embodiment, the band device includes an adjustment system configured to adapt a bracelet size to the body portion. The adjustment system may be an elastic portion, a hook and loop system or even a buckle. This buckle may be present on a lateral portion of the docking station or present on the bracelet.

According to another embodiment, the control unit includes tubing connections. For instance, the tubing connections between the pumping means and the first connection means. Advantageously, the tubing connection are made in one piece with the housing. Thus, this embodiment allows to avoid any use of flexible tubes that are more prone to tear, kinks, twists . . . . Therefore, the process to build the control unit is faster and easier to implement.

According to another embodiment, the wearable device includes a calibration station configured to calibrate at least one pressure sensor of the control unit. The pressure sensor is configured to determine the pressure in the bladder and exerted on the body portion. Thanks to this measurement, it is possible to determine the blood pressure of the subject. The calibration station is positioned between the docking station and the control unit. It allows to check the good-functioning of the pressure sensor and therefore the accuracy of the blood pressure measurement.

According to another aspect, the invention relates to a band device intended for use in a wearable device as described above, said band device co-operating with a control unit, said band device is configured to circle the body portion. The band device comprises:
a bracelet comprising a bladder configured to be inflated and deflated; and
a docking station configured to removably secure the control unit, said docking station including second connection means configured to co-operate with first connection means of the control unit, to allow said pump to inflate or deflate the bladder, and wherein a first extremity of the bracelet is connected to a first lateral portion of the docking station and a second extremity of the bracelet is connected to a second lateral portion of the docking station, said first lateral portion and second lateral portion facing each other, at least one of the first lateral portion and second lateral portion forming a connecting portion, said connecting portion including said second connection means, the docking station being interposed between the first extremity and the second extremity to close the band device.

The invention also relates to a control unit intended for use in a wearable device as previously described, said control unit co-operating with a band device, wherein the control unit comprises:
a housing;
pumping means within the housing; and
first connection means in fluidic connection with the pumping means, said first connection means being configured to allow a fluid to flow between the pumping means and an external environment of the housing,
said control unit being configured to calculate a blood pressure value using the pressure exerted by the bladder on the body portion.

### BRIEF DESCRIPTION OF THE DRAWINGS

FIG. 1 is a perspective view of the wearable device according to a first embodiment,

FIG. 2 is a top exploded view of the wearable device from FIG. 1,

FIG. 3 is a bottom exploded view of the wearable device from FIG. 1,

FIG. 4 is a longitudinal cross-sectional view of the wearable device from FIG. 1,

FIG. 5 is a perspective view of the wearable device according to a second embodiment where the adjustment system includes a buckle,

FIG. 6 is a perspective view of an embodiment of the docking station,

FIG. 7 is a top view of an embodiment of the control unit,

FIG. 8 is a perspective view of the control unit from FIG. 7,

FIG. 9 is a top view of the control unit co-operating with the docking station,

FIG. 10 is a longitudinal cross-sectional view of the control unit co-operating with the docking station from FIG. 5,

FIG. 11 is a perspective view of the co-operation between the docking station and sealing pieces,

FIG. 12 is a perspective view of the co-operation between the docking station and securing elements,

FIG. 13 is a perspective view of an embodiment of a sealing piece,

FIG. 14 is an exploded view of the control unit co-operation with a charging station,

FIG. 15 is a perspective view of the calibration station co-operating with the control unit and the docking station, and

FIG. 16 is a schematic front view of the calibration station from FIG. 15;

FIG. 17 is a schematic front view of the wearable device according to a third embodiment.

### DETAILED DESCRIPTION

The following detailed description will be better understood when read in conjunction with the drawings. For the purpose of illustrating, the wearable device is shown in the preferred embodiments. It should be understood, however that the application is not limited to the precise arrangements, structures, features, embodiments, and aspect shown. The drawings are not drawn to scale and are not intended to limit the scope of the claims to the embodiments depicted. Accordingly, it should be understood that where features mentioned in the appended claims are followed by reference signs, such signs are included solely for the purpose of enhancing the intelligibility of the claims and are in no way limiting on the scope of the claims.

The present invention relates to a wearable device 100 for measuring the vital signs and more particularly the blood pressure of a subject.

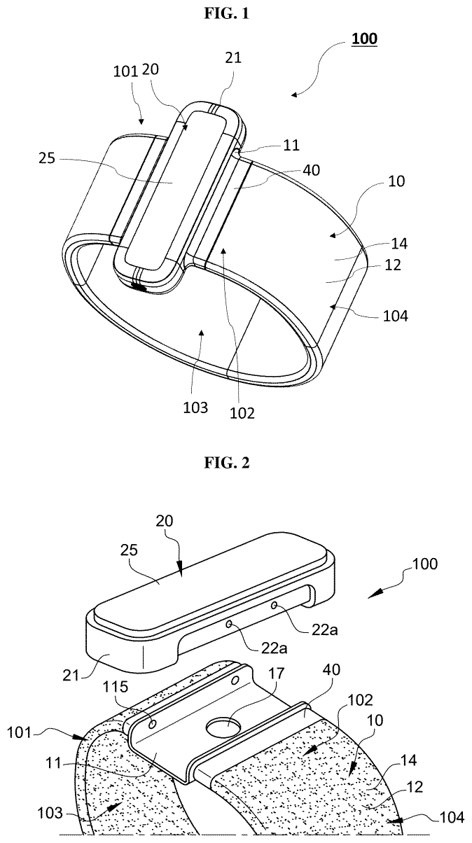

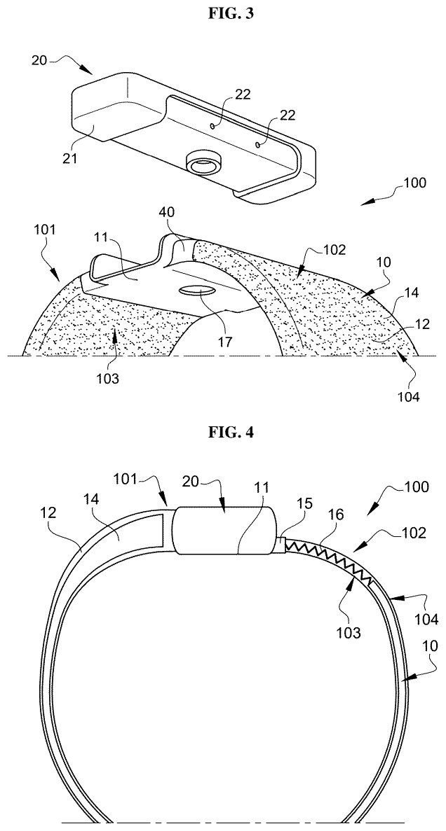

As illustrated on FIGS. 1 to 5, the wearable device 100 comprises a control unit 20 cooperating with a band device 10. The control unit 20 comprises all the electronics and pumping means gathered and protected inside a housing 21, while the band device 10 only comprises the bladder 14. The control unit 20 is removable from the band device 10 and can be attached or detached from the band device 10 at will.

### Control Unit

The control unit 20 comprises a housing 21 with a substantially parallelepiped shape. Advantageously, the housing 21 has a length for example comprised between 5 and 10 cm, a width for example comprised between 2 and 5 cm and a thickness for example comprised between 0.5 and 3 cm. Corners of the housing 21 may have a more rounded shape for aesthetic purpose. The housing 21 may be made of any kind of solid or flexible material, such as: polypropylene, ABS, PVC, Polyamide, rubber, resin, silicone, urethane, nylon, glass such as Gorilla®, or titanium.

The control unit 20 may be opened thanks to a removable lid, not represented in the figures. The removable lid may be the upper or lower wall of the housing 21. In one embodiment, the screen is removable and allows access to the inside of the housing 21.

As illustrated on FIGS. 7 and 8, the inside of the housing 21 is hollow and contains the electronics and pumping means.

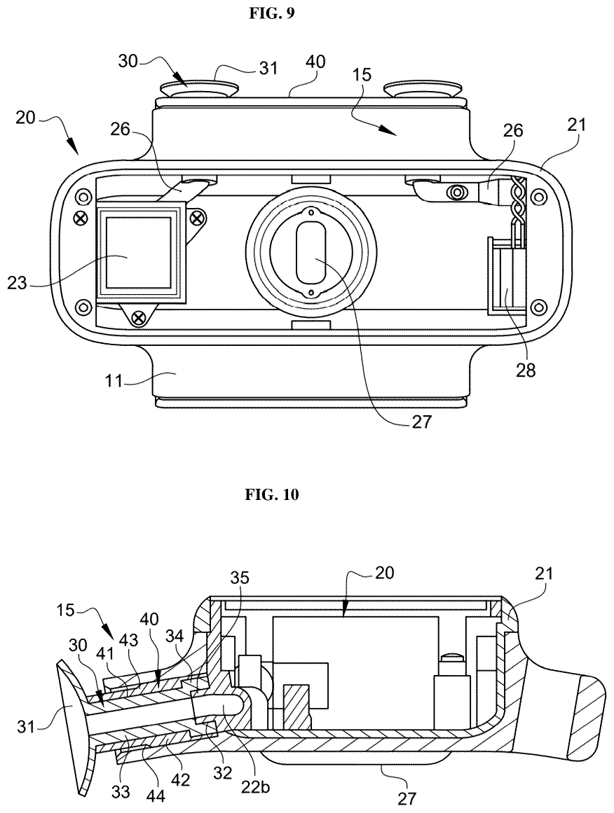

The pumping means comprise a pump 23, represented on FIG. 9, that may be a rolling air pump, a piezo electric pump, a resonant gas pump, a diaphragm/membrane gas pump, and the like.

The pump 23 is in charge of putting a fluid in motion. Preferably, the fluid is air. The pump 23 is in fluidic connection with first connection means 22, 22a, 22b. The fluid may flow in a first direction, from the first connection means 22, 22a, 22b toward the pump 23 and in a second direction, from the pump 23 toward the first connection means 22, 22a, 22b.

In practice, the first connection means 22, 22a, 22b include at least an opening localized in a wall, preferably a lateral wall, of said housing 21 and configured for the passage of the fluid between the inside of the control unit 20 and the external environment. For instance, the first connection means may be male-type first connection means 22b as illustrated on FIG. 8, meaning that they may protrude from the control unit 20. For instance, male-type first connection means 22b present a tube-like shape with a length comprised between 0.3 and 1 cm and an external diameter comprised between 0.1 to 0.5 cm. Alternatively, the first connection means may be female-type first connection means 22a, as illustrated on FIG. 2, meaning that they can either be a mere opening or protrude toward the inside of the control unit 20.

The pump 23 is connected to the first connection means 22, 22a, 22b with a network of tubing connections 26. Said tubing connections 26 may be flexible or rigid.

Moreover, the network of tubing connections 26 may be built in one piece, for instance by molding or 3D printing. The resulting piece may be fixed to the housing 21 using connection means. For instance, a frame connects the network of tubing connections 26 and said frame may be clipped on the housing 21 using a plug and socket system.

According to an embodiment, the first connection means 22, 22a, 22b include two elements of connection: a first element configured to let fluid enter the housing 21 and a second element configured to let fluid exit the housing 21. The pump 23 may be connected to both elements with a dedicated arrangement of tubing connections 26. The pump 23 is therefore configured to pump the fluid outside of the housing 21 through the first elements of connection and to pump the fluid inside the housing 21, through the second element. Alternatively, there is only one element of connection, through which the pump 23 may pump-in and out the fluid.

Both elements of the first connection means 22, 22a, 22b may be positioned on the same side of the housing 21 or alternatively, a first element may be positioned on a first lateral side of the housing 21 and the other element may be positioned on the opposite lateral side.

At least one valve, not represented in the figures, may also be included in the pumping means to control the passage of fluid through the network of tubing connections 26, for instance by opening, closing, or partially obstructing at least one of the tubing connections 26. Alternatively, a protection system could be included with a second valve in case of failure of the first valve, to ensure that there is no long-term arm compression of the patient. The valve may be physically separated from the pump 23 or integrated in the pump 23. For instance, the pump 23 may comprise one or more blowers, which allows to discharge air, thereby providing a passive valve function.

In another embodiment, the first element of the first connection means 22, 22a, 22b may be connected to the pump 23 and the other element may be connected to the valve.

As an alternative to the pump 23, the pumping means may comprise two distinct components, one of which is configured to pump the fluid in, and the other one configured to pump the fluid out.

The control unit 20 may also comprise a power supply device, not represented in the figures, configured to supply power to the components of the control unit 20. Said power supply device may comprise a built-in battery and/or removable battery. In one embodiment, the removable battery is rechargeable. In another embodiment, the built-in battery has capacity comprised between 10 mAh and 300 mAh, preferably between 30 mAh and 100 mAh. This embodiment ensures that the built-in battery duration is long enough to ensure the continuity of the functions whilst the removable battery is not in place, for instance while the removable battery is being replaced or recharged. For instance, the continuity of the functions may be ensured for at least 30 minutes; preferably for at least 40 minutes, 50 minutes or 60 minutes.

The wearable device 100 of the invention may be configured to work for the auscultatory technique or the oscillometric technique.

The first technique is based on the Korotkoff sounds recorded during the cuff inflations and deflations. In that case, the control unit 20 may not include any other feature. The medical staff may parameter the control unit 20 to inflate and deflate the bladder 14 while listening to the Korotkoff sounds with a stethoscope. Alternatively, the wearable device 100 may include a sensor configured to automatically record the Korotkoff sounds. Such sensor may be a PPG sensor for instance.

The second technique estimates the systolic and diastolic pressures based on the amplitude of the pressure oscillations recorded within the cuff during deflation or inflation. In that case, the control unit 20 preferably includes at least one pressure sensor 28. This allows to monitor the pressure in the bladder 14 and to obtain the blood pressure of the subject therefrom, via an oscillometric technique or “cuff-based” technique. More precisely, oscillometry is a technology based on the principle of occluding blood flow within the patient's limb with the inflatable cuff. Unlike the auscultation method, it measures the pulses transduced through the inflatable cuff, and exploits the fact that the pulse amplitude is modulated by the difference between the mean arterial pressure and the applied pressure in the cuff. The pulses amplitude increases when the applied pressure is between the patient's diastolic blood pressure and the mean arterial pressure, while the pulses amplitude decreases when the applied pressure is between the mean arterial pressure and the systolic blood pressure. Variation of pulses amplitude with the applied pressure yields a quasi-symmetric waveform from which the systolic and diastolic blood pressures can be determined using empirical formulas. Advantageously, the oscillometric technique allows to independently measure systolic and diastolic blood pressures. Moreover, the oscillometric technique provides an absolute blood pressure value.

In one embodiment, the wearable device 100 measures pressure during a controlled deflation of the bladder. Preferably, in another embodiment, the wearable device 100 measures blood pressure during the inflation of the bladder. This method ensures the patient's comfort because of the shorter occlusion time of the artery. It also has a reduced battery consumption.

The pressure sensor 28 may be of any type, for instance it may be a piezoresistive pressure sensor.

According to an embodiment, the pressure sensor 28 is connected to one of the first connection means 22, 22a, 22b, while the pump 23 is connected to the other first connection means 22, 22a, 22b. This configuration allows to get better pressure measurements in particular when the pump 23 is activated during the blood pressure measurement.

In another embodiment, the pump 23 and the pressure sensor 28 are connected to the same first connection means 22, 22 a, 22b. Advantageously, a protection system could be included with a second pressure sensor in case of failure of the first pressure sensor, to ensure that the patient's limb is not compressed for an extended period of time.

In more advanced sphygmomanometers, the blood pressure value may be obtained using several techniques at the same time. For instance, a first blood pressure value may be obtained using a cuff-based technique and a second blood pressure value may be obtained using an optical sensor 27. Therefore, the control unit 20 may also comprise at least one optical sensor 27. The optical sensor 27 is arranged so that it is in close contact with the subject's skin to allows to acquire a signal at an elevated acquisition frequency, which is suitable for a continuous or regular longitudinal monitoring of the subject's blood pressure.

Preferably, the optical sensor 27 is a photoplethysmographic (PPG) sensor comprising a light source and a light detector, more preferably a single-wavelength PPG sensor. For instance, the light source may be an infrared LED. In one embodiment, the light source emits an infrared radiation from about 700 nm to about 1000 nm, preferably from about 900 nm to about 980 nm. In one preferred embodiment, the light source is configured to emit an infrared radiation of about 940 nm. The infrared radiation wavelengths herein mentioned preferably are the centroid wavelengths or the peak wavelengths of the light emitters at their operating temperature. The wearable device 100 may comprise several optical sensors, which may be structurally independent or embedded within a sensor array. In one particular example, the wearable device 100 comprises at least three optical sensors and preferably: at least one infrared LED, at least one green LED, at least one red LED. Preferably, said infrared LED, green LED and red LED are configured to emit a radiation having a radiation wavelength of 940 nm, 536 nm, and 655 nm, respectively. Advantageously, the optical sensor may further comprise a light filtering element configured to minimize the intensity of the light reflected from the skin surface without affecting the light reflected by subcutaneous tissues. The light filter element may comprise a first sheet of polarized glass placed on the light source of the optical sensor and/or a second sheet of polarized glass placed on the light detector of the optical sensor.

Preferably, the housing 21 includes an opening in its inferior wall to allow the optical sensor to take its measurements. The opening is preferably made with a circular shape with a diameter for example comprised between 0.5 and 3 cm. The optical sensor 27 may protrude through the opening to come in close contact with the subject's skin.

Additionally, the control unit 20 may include other sensors such as a temperature sensor, a heat flux sensor, a position sensor, an accelerometer, a pressure transducer, an ultrasound sensor, an accelerometer, a gyroscopic sensor, an electrical sensor, such as for instance an electrical sensor comprising electrodes, a tomographic sensor, an acoustic sensor, a magnetometer, a humidity sensor, or even an impedance sensor. They may be used to calculate parameters such as the oxygen saturation, the heart rate, the respiratory rate, the blood pressure trend, the heart rate variability, the cardiac output, the skin temperature, the body temperature, or even the body composition.

As illustrated in FIG. 1 or 2, a top external part of the housing 21 may be covered by a screen 25 to allow visualization of information such as the subject's blood pressure or other vital signs measured by the wearable device 100. The screen 25 may be a liquid-crystal display (LCD), organic light emitting diode (OLED) display, light emitting diode (LED) display, a e-ink display, or the like. The housing 21 may also include button and/or switches on its external surface to power on/off the wearable device 100 or to configure the wearable device 100. Alternatively, the screen 25 may be touch-sensitive.

### Band Device

The band device 10 is configured to be worn around a body portion of the subject, such as a limb or the torso and preferably around an arm of the subject, and more particularly around the upper arm. When worn around the upper arm, the wearable device 100 is less sensitive to motion artifacts compared to wearable devices worn around the wrist or a finger. Moreover, the upper arm is characterized by elevated vascularization, and presence of main arteries such as the brachial artery. Therefore, the blood pressure measured with the present wearable device 100 is indicative of the blood pressure in the arm arteries, such as the brachial or humeral artery, which is close to the blood pressure in the aorta. This allows to provide a highly accurate and non-invasive blood pressure estimation.

As illustrated on FIGS. 1 to 4, the band device 10 comprises a docking station 11 connected to a bracelet 12 including a bladder 14.

The bracelet 12 may be obtained using two bands of fabric sawn together to form a sleeve where the bladder 14 can be fitted. The bracelet fabric may be a nylon or synthetic fiber fabric, which protects the bladder 14 from cuts during use by on-scene rescue technicians and reduces patient discomfort. The bracelet fabric is preferably very flexible to adapt to the bladder 14 inflations and deflations, durable and waterproof to resist numerous uses.

Alternatively, the bladder 14 may be directly exposed and not covered with a protective sleeve. In that case, the bladder 14 is preferably made of a thicker material to withstand wear.

The bracelet 12 has preferably a length for example comprised between 20 cm and 42 cm, preferably between 20 cm and 36 cm or between 20 cm and 30 cm. The bracelet 12 has a width that may be comprised between 5 cm and 16 cm, preferably between 9 cm and 14 cm.

An inner surface 103 and an outer surface 104 of the bracelet 12 may be defined, the inner surface 103 being configured to be in contact with the skin of the subject when the wearable device 100 is worn. A first extremity 101 of the bracelet 12 may be permanently connected to the docking station 11, while the second extremity 102 may be used to adjust the size of the bracelet 12 to the body portion size.

In one embodiment, the second extremity 102 is permanently connected to the docking station 11 or to the control unit 20 and the adjustment system includes an elastic portion 16 such as illustrated in FIG. 4. Thus, the wearable device 100 may be put on or removed by deforming the elastic portion 16.

In another embodiment, the second extremity 102 may be removably connected to the docking station 11 or to the control unit 20, thus allowing to open the bracelet 12 to put on or remove the wearable device 100 from around the body portion.

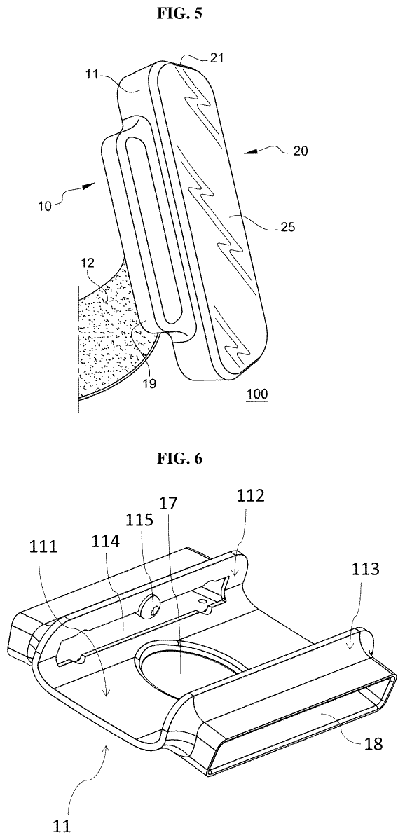

As illustrated on FIG. 5 and FIG. 11, the adjustment system may include a buckle 19 formed on a lateral portion of the docking station 11 or on a lateral portion of the control unit 20 and fixation means such as a hook and loop system or snap fasteners may be present on the second extremity 102 of the bracelet 12. The second extremity 102 can be threaded through the buckle 19 and fastened on itself to adjust the size of the bracelet 12.

As aforementioned, a bladder 14 is included between the inner surface 103 and outer surface 104. The bladder 14 is inflatable to a pressure that is high enough to uniformly restrict the blood flow in the subject's arm around which the bracelet 10 is worn. In other words, the bladder 14 may be inflatable up to a maximum inflation pressure which is superior to the pressure needed to occlude the brachial artery. Said maximum inflation pressure may be equal to 250 mm Hg, preferably 260 mm Hg, 270 mm Hg or 280 mm Hg. In some cases, it may be desirable to provide a bladder 14 inflatable up to a pressure of 300 mm Hg.

According to one embodiment illustrated on FIG. 4, the inflatable bladder 14 may be made of a single inflatable chamber that does not extend throughout the length of the bracelet 12. For instance, the inflatable bladder 14 may extend for a length between 20 and 42 cm preferably between 22 cm and 36 cm., so as to cover for instance 80% of the wearer's arm. Alternatively, the bladder 14 may extend throughout the length of the bracelet 12. Alternatively, the bladder 14 may contain several inflatable chambers distributed along the bracelet 12.

The bladder 14 may be made of resin, rubber, polyester, synthetic fiber, or a combination thereof. Preferably, the bladder 14 is made of plastic fibers, TPU, PVC, PA or a combination thereof. In some embodiments, the bladder 14 may be made of coated fabrics such as Nylon™.

The bladder 14 includes an opening in which a tubing connection may be fitted.

The band device 10 may present a closed state wherein the band device 10 circles the body portion of the subject. In other words, the closed state corresponds to the configuration of the wearable device 100 wherein the control unit 20, the docking station 11 and the bracelet 12 cooperate mechanically and fluidically to form a continuous circle around the body portion. In other words, the bracelet 12 only partially circles the body portion. The remaining portion is completed by the presence of the docking station 11 and optionally the control unit 21, when the docking station 11 has a length inferior to a length of the housing 21 bottom wall or when the docking station 11 is positioned on a lateral wall of the housing 21. In that state, the wearable device 100 is ready to be used to measure blood pressure by inflating the bladder 14.

Additionally, the band device 10 may present an open state corresponding to a configuration of the wearable device 100 wherein the docking station 11 and/or the control unit 20 is detached from the bracelet 12 in such a way that it is possible for the subject to position the wearable device 100 around his body portion before closing the band device 10.

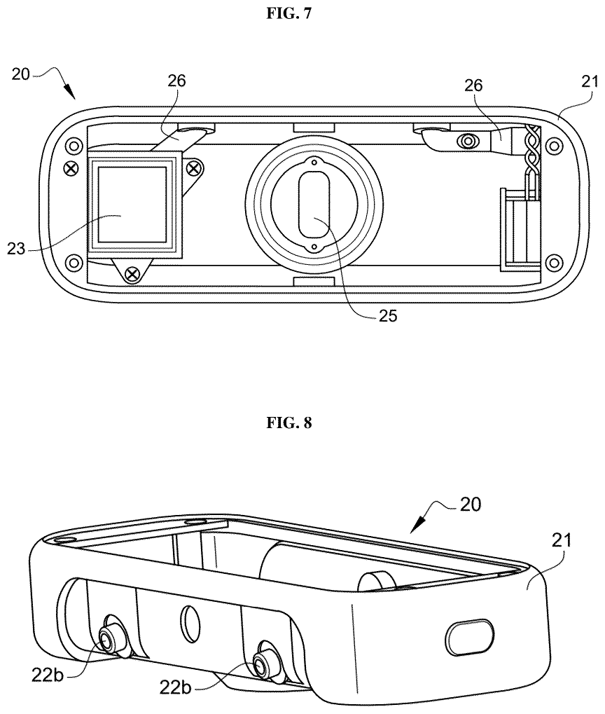

As illustrated on FIG. 6, the docking station 11 comprises a bottom wall 111 configured to come in contact with the skin, framed by at least one lateral wall 112, 113, thus forming together a reception site configured to receive the control unit 20. A first lateral wall 112 of the docking station 11 may define a first lateral portion and a second lateral wall 113 or the docking station 11 may define a second lateral portion. Alternatively, the first lateral portion may also be defined by a lateral wall of the control unit 20.

For instance, the reception site may have a complementary shape to the control unit 20. Therefore, the reception site may have a parallelepiped shape with a length for example comprised between 5 and 10 cm, a width for example comprised between 2 and 5 cm and a thickness for example comprised between 0.5 and 3 cm.

Alternatively, the reception site may encapsulate only a portion of the control unit 20. For instance, the reception site may have a bottom wall 111 with a length inferior to a length of the housing 21 bottom wall. FIG. 17 illustrates an embodiment wherein the reception site comprises a bottom wall 111 with a length inferior to a length of the housing 21 bottom wall and a lateral wall 112 comprising the second connection means configured to establish the fluidic connection between the pump 23 and the bladder 14.

FIG. 17 further illustrates a closed state wherein the control unit 20 cooperates with the first extremity 101 of the bracelet 12 to close the wearable device 100. For that purpose, the control unit 20 may include an adjustment system configured to adapt a bracelet size to the body portion. The adjustment system may be an elastic portion, a hook and loop system or even a buckle. This buckle may be present on a lateral portion of the control unit 20.

Alternatively, the reception site of the docking station 11 may only cover a lateral wall of the control unit 20. In that case, the docking station only comprises a lateral wall 112 comprising the second connection means configured to establish the fluidic connection between the pump 23 and the bladder 14.

### Cooperation Between the Control Unit and the Band Device

The control unit 20 may be plugged and unplugged on the band device 10 at will. To that end, the control unit 20 may be fitted inside the reception site of the docking station 11 in several ways. For instance, the control unit 20 may be inserted by a movement from top down. Alternatively, the control unit 20 may be inserted by a sliding movement from one side to the other. In that case, the control unit 20 may include at least one rail cooperating with at least one slide on the docking station side walls 112, 113.

Once installed on the docking station 11, the control unit 20 may either extend over the ends of the docking station 11 or be completely enclosed inside the reception so that it is protected from chocs and cannot be easily dislodged.

The control unit 20 may be maintained in the reception site using fixing means, such as deformable ergots 115, as illustrated in FIG. 6, or a plug and socket system. Alternatively, the fixing means is a system of clip with male ergots on the reception site and female ergots on the control unit 20. Moreover, the control unit 20 may present slots to receive such fixing means.

Advantageously, the docking station 11 comprises a through hole 17 in its bottom wall 111. The through hole 17 is configured to allow insertion of a sensor such as an optical sensor 25 of the control unit 20. Preferably, the through hole 17 is made with a circular shape with a diameter comprised between 0.5 and 3 cm. The through hole 17 in the docking station bottom wall 111 is configured to be aligned with the opening of the control unit 20 to allow the optical sensor 25 to protrude through the through hole 17 and come in close contact with the subject's skin.

The connection between the control unit 20 and the band device 10 and more precisely between the pump 23 and the bladder 14 is ensured by the association of several elements. In the end, the first connection means 22, 22a, 22b from the control unit 20 have to be put in fluid-proof contact with the second connection means 15 of the docking station 11.

For that purpose, the docking station 11 comprises a connecting portion corresponding to at least one of the first lateral portion and second lateral portion of the docking station 11, said connecting portion including the second connection means 15. On FIG. 6, the connecting portion corresponds to the first lateral wall 112 in which an opening 114 is created. Moreover, the external surface of the first lateral wall 112 forms a recess 18. This arrangement is configured to receive a first securing element 40 and at least one sealing element 30.

### Securing Element

The securing element 40 is illustrated in FIGS. 9 to 12. The securing element 40 is configured to be fitted in the recess 18. In the illustrated embodiment, the securing element 40 has a parallelepiped shape with a length for example comprised between 5 and 15 cm, a width for example comprised between 2 and 5 cm and thickness for example comprised between 0.5 and 1.5 cm. It comprises at least one opening that crosses along its entire length, the opening being configured to receive the body of a sealing element 30. Advantageously, the securing element 40 is made of two parts: an upper part 41 and a lower part 42. Both parts cooperate to form the opening configured to receive the body of the sealing element 30. For instance, the upper part 41 and lower part 42 may comprise a slot crossing along their respective length, so that when the two parts are assembled, the opening configured to receive the sealing element 30 is formed. The opening has preferably a cylindrical shape and the slots may have a hemi-cylindrical shape, so that when the upper part 41 and a lower part 42 are assembled, the cylindrical opening is formed.

The securing element 40 may have two of such openings, in order to fit two sealing elements 30. One sealing element 30 being configured for the passage of a fluid in a first direction and the other sealing element being configured for the passage of a fluid in the opposite direction. Alternatively, the securing element 40 may only have one opening, in order to fit a single sealing element 30 configured for the passage of a fluid in both directions.

The upper part 41 and lower part 42 may be fixed together using fixing elements such as screws and bolts.

The securing element 40 may present notches 43, 44 on the outer face of the upper part 41 and lower part 42. The notches 43, 44 are designed to create an insertion recess configured for the insertion of the bracelet inner surface and an outer surface fabric.

### Sealing Elements

The sealing element 30 is preferably made of any material exhibiting elastic or rubber-like properties, such as polymers, neoprene, ethylene-vinyl acetate, nitrile, Closed Cell Silicone Sponge, ABS, polycarbonate (Lexan, Makrolon), polyethylene (LLDPE, LDPE, MDPE, HDPE, HMW-HDPE), polypropylene (homopolymer, copolymer), polystyrene (HIPS, crystal styrene), polyurethane (polyester, polyether), PVC (rigid, flex), synthetic rubber (TPV, TPU, TPE, OBC). Therefore, the sealing element 30 can easily be deformed to be inserted in openings or to fit around connection elements.

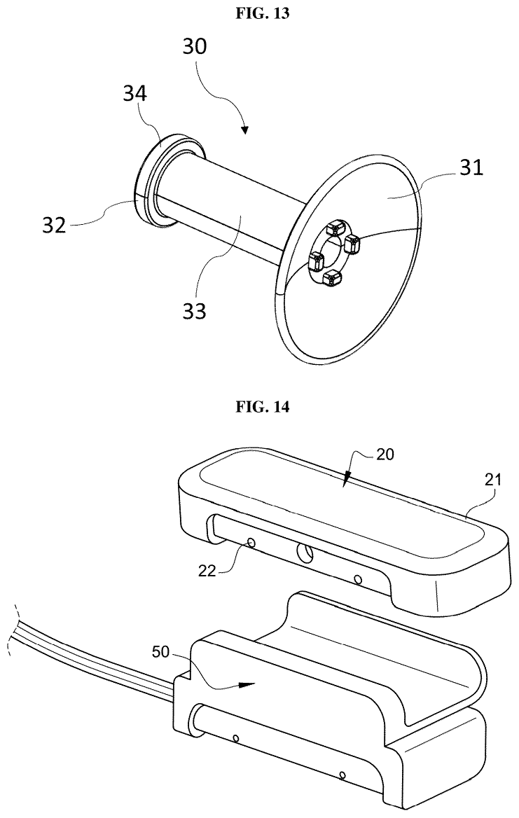

As illustrated in FIG. 13, the sealing element 30 comprises a tube-shaped body 33 and two ends. The body 33 is hollow and can be crossed through by a fluid. The external diameter of the body 33 is comprised for example between 0.1 and 1 cm and the internal diameter of the body 33 is comprised for example between 0.05 and 0.8 cm. The length of the whole sealing element 30 is comprised for example between 2 and 5 cm.

The first end 31 of the sealing element 30 may have a flaring shape, meaning that the diameter of the sealing element 30 increases at its first end 31. For instance, the flaring shape corresponds to a shoulder, meaning that the diameter increases radially such as illustrated in FIG. 13. Hence, the external diameter of the first end 31 is comprised for example between 0.2 and 1 cm and the internal diameter of the tube is comprised for example between 0.02 and 0.8 cm. Alternatively, the diameter may increase gradually, thus forming a paraboloid shape. Either way, the first end 31 forms contact surfaces 35, 36 configured to guarantee fluid-proofness of the connection.

For instance, the first end illustrated in FIG. 10 is in the form of a shoulder. When the control unit 20 is fitted in the docking station reception site, the male-type first connection means 22b crosses through the opening 114 in the lateral wall 113. The cooperation between the male-type first connection means 22b and the first end 31 is obtained by inserting in force the male-type first connection means 22b inside the first end 31 opening. The inner diameter of the first end 31 forms an inner contact surface 35 for the male-type first connection means 22b, while the transversal part of the shoulder portion forms an outer surface 36. This outer surface 36 is configured to come in contact with a side wall of the securing element 40. The securing element 40 maintains the first end 31 around the male-type first connection mean 22b of the control unit 20. The first end 31 is therefore sandwiched and maintained into place between the securing element 40 and the control unit 20, thus ensuring fluid-proofness of the connection.

According to another example, the first connection means is a female-type first connection mean 22a such as illustrated in FIG. 2. In that case, the first end, not represented in the figures, may have a flaring shape in the form of a paraboloid where the apex is directed toward the extremal part of the first end, while the base is directed toward the second end 32. The cooperation between the female-type first connection means 22a and the first end is obtained by inserting in force the first end inside the female-type first connection mean 22a opening. When inserted, the paraboloid shape is deformed to fit in the opening. Once the base of the paraboloid has crossed the opening, it can deploy back to its original shape. Therefore, the base of the paraboloid forms a contact surface of bigger diameter than the opening diameter. Said base can be pressed against the opening of the female-type first connection means 22a, thus ensuring fluid-proofness of the connection. Therefore, the fluid can transit between the network of tubing connections 26 and the body 33 without any leak.

The second end 32 of the sealing element 30 also has a flaring shape, meaning that the diameter of the sealing element 30 increases at its second end 32. Advantageously, the diameter of the second end 32 increases gradually, thus forming a paraboloid shape where the base is directed toward the extremal part of the second end 32. The diameter may for instance increase from 0.2 cm to 3 cm.

In use, the second end 32 is inserted inside the bladder 14 by deforming the flaring shape. Once inserted, the deformed second end 32 reverts to its original flaring shape and can be pressed against the opening of the bladder 14 to ensure fluid-proofness of the connection. Alternatively, screws or a clip system may be used to ensure fluid-proofness of the connection.

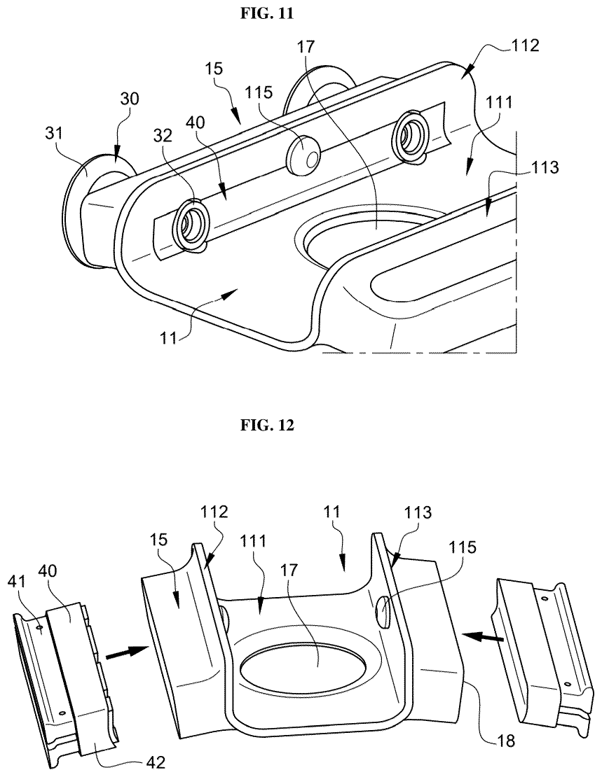

When mounting the wearable device 100, the sealing elements 30 are first fitted inside said securing element 40 so that both the first end 32 and the second end 31 extend over the edges of the securing element 40. Said sealing 30 element can either be inserted in force inside the securing element 40 opening by a deformation of its body or the upper part 41 and lower part 42 may be closed around the sealing element body 30. Then the second end 31 is inserted inside the bladder 14. The inner surface 103 and outer surface 104 fabric of the bracelet 12 are then fitted against the sides of the recess 18 and the securing element 40 is inserted in the recess 18, as illustrated in FIG. 12, on one side of the docking station 11, thus blocking the inner surface 103 and an outer surface 104 fabric of the bracelet 12 in the notches 43, 44, between the recess walls and the securing element 40.

On the other side of the docking station 11, the inner surface 103 and outer surface 104 fabric of the opposite end of the bracelet 12 may also be fitted against the walls of a second recess 18. A second securing element 40, with no sealing element 30 inside, may be inserted in said recess 18, thus blocking the inner surface 103 and an outer surface 104 fabric.

In another embodiment, the other side of the docking station 11 has no recess 18 and a buckle 19 is present instead, thus allowing to attach the other end of the bracelet 12.

Lastly, the control unit 20 is fitted inside the reception site of the docking station 11, either by first inserting the male-type first connection means 22b in the first end 31 of the sealing element 30 or by plugging the first end 31 inside the female-type first connection means 22a.

Alternatively, the connection between the docking station 11, the bracelet 12 and the housing 21 may be a removable connection, meaning that the docking station 11 and the bracelet may be plugged together and unplugged at will in the same way that the housing 21 may be plugged and unplugged at will from the docking station 11. To that end any plug/unplug connector known from the man skilled in the art may be used. For instance, quick-connect couplings may be used for joining components with an airflow requirement. These couplings encompass a male and a female component that seamlessly interlock. When engaged, they create a sealed conduit for air to traverse. Threaded connections involve the act of screwing one part into another. These are commonly employed with threaded pipes and fittings, permitting airflow while connected. Push-to-connect fittings employ a mechanism where one part is pushed into another, forming a secure connection with airflow capability. Bayonet connections involve the alignment and twisting of one part into another, enabling airflow between the connected components. Camlock couplings utilize a lever or cam mechanism to secure the connection, facilitating airflow passage. Luer lock connectors are commonly utilized in medical applications for connecting components with airflow needs. They employ a twist-lock mechanism to establish a secure connection that permits the passage of air. Magnetic connectors employ magnets to secure two parts together while maintaining an airflow channel. Snap-fit connections involve interlocking one part into another, creating an airtight seal that allows airflow. Slide and lock connections require sliding one part into the other and locking them in place, enabling the passage of air. Buckle or clamp mechanisms may be employed to securely fasten two parts with airflow requirements. A bay connector with a pressure plate mechanism enables the flow of air when pressed together. Some connectors may also require a twist-and-pull method for plugging and unplugging while allowing airflow.

### Calibration Station

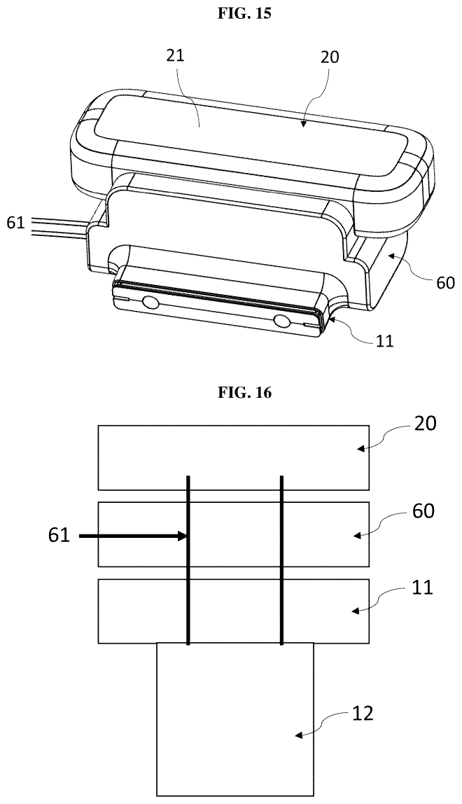

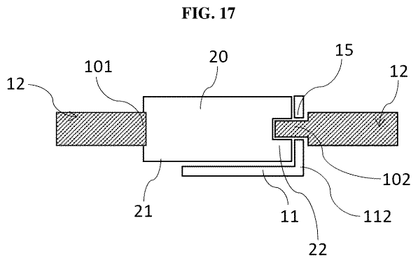

Advantageously, the wearable device 100 includes a calibration station 60, as illustrated in FIGS. 15 and 16. The calibration station 60 is used to test the wearable device 100 and to verify if it meets safety standards such as the ISO standards IEC 80601-2-30 on medical electrical equipment.

For this purpose, the calibration station 60 may check different aspects of the wearable device 100. For instance, it checks if the pressure sensor 28 is accurate using a simulator 61. It checks if there are any leaks problems in the wearable device 100. It performs “static pressure” tests. It may also check that the safety modes work well. Such safety modes may be an automatic deflation in the event of overpressure. For instance, the safety mode may prevent the pressure in the bladder 14 from exceeding a maximum value such as the one specified in the ISO standards IEC 80601-2-30, clause 201.12.1.104, which is of 300 mmHg, by more than 10 %, for more than 3 seconds. As another example, the safety mode may prevent the pressure in the bladder 14 from exceeding a maximum value such as the one specified in the ISO standard 201.12.1.104 for more than 15 seconds. When activated, the safety mode deflates the bladder 14 within 30 seconds to a pressure value inferior to 15 mmHg.

As illustrated in FIG. 16, the calibration station 60 may be interposed between the control unit 20 and the docking station 11. The calibration station 60 may comprise an upper side framed by two lateral walls comprising connection means configured to cooperate with the first connection means 22, 22a, 22b. The upper side and lateral walls form a reception site configured to receive the control unit 20.

The calibration station 60 may also include a lower side comprising connection means configured to fit in the docking station 11 reception site and to cooperate with the second connection means 15. Therefore, when the calibration station 60 is interposed between the control unit 20 and the docking station 11, it ensures connection of the pumping means with the bladder 14, through the calibration station 60, thus making it possible to perform the different tests.

To conclude, the invention allows to obtain a modular device with an easy-to-use cooperation system between the different available control units and different band devices. The wearable device is very adaptable to patients with different needs and morphologies.

### Kit

The wearable device 100 is configured so that it is possible to connect a range of different control unit 20 with different features on the band device 10. In the same manner, the wearable device 100 is also configured to allow the connection of band devices 10 with different features to the control unit 20.

For instance, it is possible to have a kit with a control unit 20 and several band devices 10 with different sizes of bracelet 12. For instance, a first band device 10 may have a bracelet 12 with a length of 15 cm dedicated to children, a bracelet 12 with a length of 25 cm and a bracelet 12 with a length of 35 cm.

As another example, the kit may comprise a band device 10 with several control units 20. The control unit 20 may be chosen depending on the patient's need. A first control unit 20 may include only sensors for taking a blood pressure value. A second control unit 20 may include a position sensor and/or an accelerometer to also detect the patient's movements during the day and correlate the with the blood pressure variations. A third control unit 20 may include additional sensors to measure other vital signs such as the oxygen saturation, the heart rate, the respiratory rate and the body temperature for a comprehensive monitoring of the patient.

Such as illustrated in FIG. 14, the kit may also include a charging station 50 to charge the control unit 20 battery. The control unit 20 may cooperate with the charging station 50 in the same way than with the band device 10. As such, the control unit 20 may be fitted inside the reception site of the charging station 50 by a movement from top down. Alternatively, the control unit 20 may be inserted by a sliding movement from one side to the other. In that case, the control unit 20 may include a rail cooperating with a slide on the charging station side walls.

1-15. (canceled)

16. A wearable device for measuring blood pressure of a subject comprising:
a control unit comprising:
a housing;
pumping means within said housing; and
first connection means in fluidic connection with the pumping means, said first connection means being configured to allow a fluid to flow between the pumping means and an external environment of the housing;
a band device configured to circle a body portion of the subject when in a closed state, said band device comprising:
a bracelet comprising a bladder configured to be inflated and deflated to exert a pressure on the body portion; and
a docking station configured to removably secure the control unit, said docking station including second connection means configured to co-operate with the first connection means to allow said pumping means to inflate or deflate the bladder,
wherein said control unit is configured to determine blood pressure using the pressure exerted by the bladder on the body portion, wherein a first extremity of the bracelet is connected to a first lateral portion of either one of the docking station and the control unit, and a second extremity of the bracelet is connected to a second lateral portion of the docking station, said first lateral portion and second lateral portion facing each other, at least one of the first lateral portion and second lateral portion forming a connecting portion, said connecting portion including said second connection means, the docking station being interposed between the first extremity and the second extremity in the closed state.

17. The wearable device according to claim 16, wherein said second connection means are further configured to attach at least one of the first extremity and second extremity of the bracelet to the docking station.

18. The wearable device according to claim 16, wherein said second connection means include at least one sealing element comprising:
a tube-shaped body configured to cross through the connecting portion;
a first end configured to co-operate with the first connection means to establish a fluid-proof connection; and
a second end configured to co-operate with the bladder to establish a fluid-proof connection.

19. The wearable device according to claim 18, wherein said connecting portion further includes at least one securing element, said securing element and said sealing element being configured to at least partially be inserted in a recess arranged in the connecting portion.

20. The wearable device according to claim 19, wherein said securing element comprises an upper part and a lower part, said upper part and lower part being configured to co-operate to accommodate the tube-shaped body of the sealing element and to maintain one of the first extremity and second extremity.

21. The wearable device according to claim 18, wherein the first end forms inner and outer contact surfaces, said first end being arranged between the first connection means and the securing element, the first connection means coming in contact with the inner contact surface and the securing element coming in contact with the outer contact surface.

22. The wearable device according to claim 18, wherein said first end forms a contact surface configured to be maintained inside said first connection means.

23. The wearable device according to claim 16, wherein said band device includes an adjustment system configured to adapt a bracelet size to the body portion.

24. The wearable device according to claim 16, wherein said housing comprises a bottom side, said bottom side including a sensor, said sensor being configured to come in contact with the body portion.

25. The wearable device according to claim 16, wherein said docking station comprises a bottom wall configured to come in contact with the skin, framed by two lateral walls comprising the second connection means, said bottom wall and lateral walls forming a reception site configured to receive the control unit.

26. The wearable device according to claim 9, wherein said docking station comprises a through hole in the bottom wall of the docking station, said through hole being configured to allow insertion of said sensor.

27. The wearable device according to claim 16, wherein the pumping means include tubing connections, said tubing connections being made in one piece with the housing.

28. The wearable device according to claim 16, wherein the device includes a calibration station configured to calibrate at least one pressure sensor of the control unit, said calibration station being positioned between the docking station and the control unit.

29. A band device intended for use in a wearable device according claim 16, said band device co-operating with a control unit, wherein said band device is configured to circle the body portion, said band device comprising:
a bracelet comprising a bladder configured to be inflated and deflated; and
a docking station configured to removably secure the control unit, said docking station including second connection means configured to co-operate with first connection means of the control unit, to allow said pump to inflate or deflate the bladder, and wherein a first extremity of the bracelet is connected to a first lateral portion of the docking station and a second extremity of the bracelet is connected to a second lateral portion of the docking station, said first lateral portion and second lateral portion facing each other, at least one of the first lateral portion and second lateral portion forming a connecting portion, said connecting portion including said second connection means, the docking station being interposed between the first extremity and the second extremity to close the band device.

30. A control unit intended for use in a wearable device according to claim 16, said control unit co-operating with a band device, wherein said control unit comprises:
a housing;
pumping means within the housing; and
first connection means in fluidic connection with the pumping means, said first connection means being configured to allow a fluid to flow between the pumping means and an external environment of the housing,
said control unit being configured to calculate a blood pressure value using the pressure exerted by the bladder on the body portion.
